(19) United States
(12) Patent Application Publication
(10) Pub. No.: US 2013/0085366 A1
(43) Pub. Date: Apr. 4, 2013

(54) BIOLOGICAL SIGNAL DETECTION ELECTRODE AND BIOLOGICAL SIGNAL DETECTION APPARATUS

(71) Applicants: Takuro Yamamoto, Kanagawa (JP); Haruhiko Soma, Tokyo (JP); Seiji Wada, Kanagawa (JP); Yusaku Nakashima, Tokyo (JP); Takashi Tomita, Kanagawa (JP)

(72) Inventors: Takuro Yamamoto, Kanagawa (JP); Haruhiko Soma, Tokyo (JP); Seiji Wada, Kanagawa (JP); Yusaku Nakashima, Tokyo (JP); Takashi Tomita, Kanagawa (JP)

(73) Assignee: Sony Corporation, Tokyo (JP)

(21) Appl. No.: 13/616,429

(22) Filed: Sep. 14, 2012

(30) Foreign Application Priority Data
Oct. 3, 2011 (JP) 2011-219418

(51) Int. Cl. G01N 27/327; A61B 5/04

(52) U.S. Cl. 600/390; 204/403.01

(57) ABSTRACT

A biological signal detection electrode includes a water-containing member and an absorbent sheet. The water-containing member is impregnated with conductive fluid and has flexibility. The absorbent sheet covers the water-containing member and the conductive fluid is capable of permeating therethrough.

### BACKGROUND

The present disclosure relates to a biological signal detection electrode that comes into contact with a surface of a biological body and detects a biological signal and a biological signal detection apparatus including the biological signal detection electrode.

A biological signal (e.g., brain wave or electromyographic signal) generated in a biological body (animals including humans) can be measured by an electrode to be connected to a body surface of the biological body. It is necessary to electrically connect the electrode and the body surface sufficiently to measure the biological signal accurately. In this regard, a method of connecting the electrode and the body surface via conductive fluid is generally employed, for example.

In Japanese Patent Application Laid-open No. 2006-006666 (paragraph [0042], FIG. 3), an elastic member formed of a sponge or the like, which is impregnated with an electrolyte solution, is used as a brain wave detection electrode. When the electrode comes into contact with a head surface and is elastic-deformed, the electrolyte solution seeps, thereby ensuring conduction between the head surface and the electrode.

### SUMMARY

However, in the electrode disclosed in Japanese Patent Application Laid-open No. 2006-006666 (paragraph [0042], FIG. 3), the electrolyte solution impregnated into the elastic member is considered to evaporate with elapse of time. Therefore, it is considered to be difficult to use the electrode for a long-time measurement. Moreover, for the long-time measurement, a wear feeling (e.g., having no pain) of the electrode is also important.

In view of the circumstances as described above, there is a need for a biological signal detection electrode that is suited for a long-time wearing and a biological signal detection apparatus.

According to an embodiment of the present disclosure, there is provided for a biological signal detection electrode including a water-containing member and an absorbent sheet.

The water-containing member is impregnated with conductive fluid and has flexibility.

The absorbent sheet covers the water-containing member and the conductive fluid is capable of permeating therethrough.

According to this configuration, since the water-containing member is deformed or displaced along a shape of a surface of a biological body, the biological signal detection electrode can be stably brought into contact with the biological body. In addition, since the absorbent sheet enables the conductive fluid to evaporate slowly, it is possible to prevent the conductive fluid from drying even if the biological signal detection electrode is used for a long time. Moreover, when the biological signal detection electrode is removed from the surface of the biological body after a measurement is completed, it is possible to remove dirt on the surface of the biological body, which is caused due to the conductive fluid, because the absorbent sheet absorbs, to some extent, the conductive fluid attached to the surface of the biological body.

The biological signal detection electrode may further include a container that is formed of a side surface and a bottom surface and includes an electrode terminal having conduction to the bottom surface. The water-containing member may be housed in the container, and the absorbent sheet may cover the container together with the water-containing member.

According to this configuration, the water-containing member can be held by the container.

The water-containing member may be formed of a plurality of beads.

According to this configuration, since the plurality of beads are moved together along the shape of the surface of the biological body, the biological signal detection electrode can be stably brought into contact with the biological body.

The water-containing member may be formed of a superabsorbent polymer.

According to this configuration, since the superabsorbent polymer is deformed along the shape of the surface of the biological body, the biological signal detection electrode can be stably brought into contact with the biological body.

The water-containing member may be formed of a plurality of beads arranged on the bottom surface of the container and a plurality of cotton applicators arranged on the plurality of beads.

According to this configuration, since the plurality of cotton applicators stretch while moving on the plurality of beads along the shape of the surface of the biological body, the biological signal detection electrode can be stably brought into contact with the biological body.

The absorbent sheet may be formed of a polyolefin film.

The water-containing member may be formed of a cross-linked polyacrylic acid partial sodium salt.

According to an embodiment of the present disclosure, there is provided for a biological signal detection apparatus including a biological signal detection electrode and a brace.

The biological signal detection electrode includes a water-containing member that is impregnated with conductive fluid and has flexibility and an absorbent sheet that covers the water-containing member and through which the conductive fluid is capable of permeating.

The brace is configured to bring the biological signal detection electrode into contact with a biological body.

As described above, according to the embodiments of the present disclosure, it is possible to provide a biological signal detection electrode that is suited for a long-time wearing and a biological signal detection apparatus.

These and other objects, features and advantages of the present disclosure will become more apparent in light of the following detailed description of best mode embodiments thereof, as illustrated in the accompanying drawings.

### BRIEF DESCRIPTION OF DRAWINGS

FIG. 1 is a perspective view showing a biological signal detection electrode according to a first embodiment of the present disclosure;

FIG. 2 is a perspective view showing a container of the biological signal detection electrode;

FIG. 3 is a perspective view showing beads housed in the container of the biological signal detection electrode;

FIG. 4 is a perspective view showing a biological signal detection electrode according to a second embodiment of the present disclosure;

FIG. 5 is a perspective view showing a biological signal detection electrode according to a third embodiment of the present disclosure;

FIG. 6 is a perspective view showing a biological signal detection electrode according to a fourth embodiment of the present disclosure;

FIG. 7 is a perspective view showing a biological signal detection electrode according to a fifth embodiment of the present disclosure; and

FIG. 8 is a perspective view showing a biological signal detection electrode according to a sixth embodiment of the present disclosure.

### DETAILED DESCRIPTION OF EMBODIMENTS

### First Embodiment

A biological signal detection electrode according to a first embodiment of the present disclosure will be described.

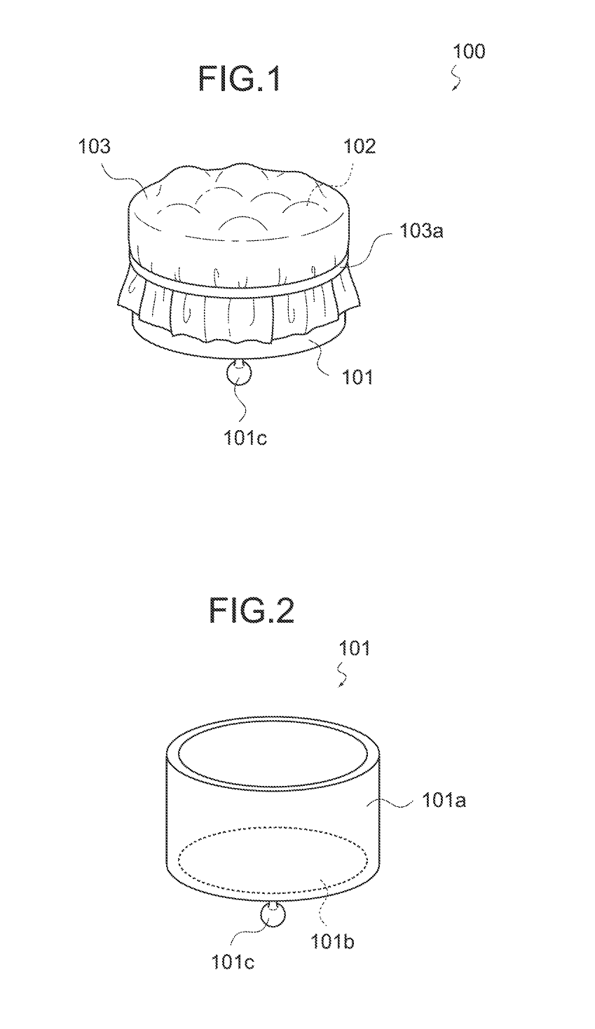

FIG. 1 is a perspective view showing a biological signal detection electrode 100 according to this embodiment.

As shown in the figure, the biological signal detection electrode 100 includes a container 101, beads 102, and an absorbent sheet 103.

The beads 102 are housed in the container 101 and covered with the absorbent sheet 103. The absorbent sheet 103 is bound to the container 101.

FIG. 2 is a perspective view showing only the container 101. As shown in the figure, the container 101 has a side surface 101a having a cylindrical shape and a bottom surface 101b that leads to the side surface 101a, and the container 101 is formed of the side surface 101a and the bottom surface 101b. It should be noted that the shape of the container 101 is not limited to the cylindrical shape.

The side surface 101a may be formed of an insulation material, e.g., synthetic resin. The bottom surface 101b may be formed of a conductive material such as metal and is configured to have conduction inside and outside the container 101. It should be noted that, on the bottom surface 101b, a terminal 101c for connecting the biological signal detection electrode 100 to a brace (e.g., headgear) is formed. Although the size of the container 101 is not particularly limited, it may be about 1 cm in diameter, for example.

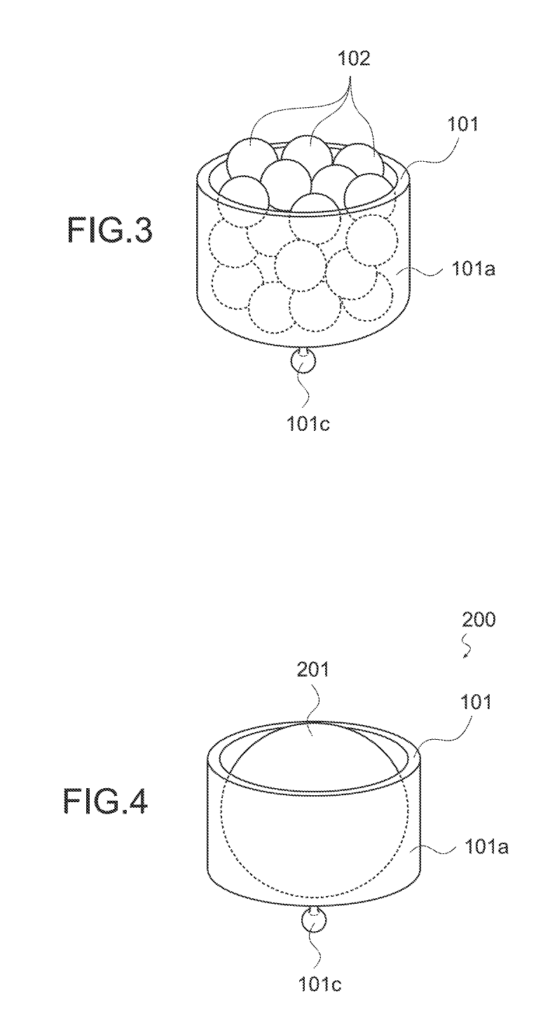

FIG. 3 is a perspective view showing the beads 102 housed in the container 101. As shown in the figure, the beads 102 are formed in size capable of being housed in the container 101 and are housed in the container 101 when being used. The beads 102 are housed in the container 101 to protrude a little from the side surface 101a of the container 101. The beads 102 may be formed of a water-retentive material capable of retaining a liquid, e.g., a cross-linked polyacrylic acid partial sodium salt.

The beads 102 are used by being impregnated with conductive fluid. Although the conductive fluid is not particularly limited, it may be sodium chloride (0.9%), for example. The conductive fluid is impregnated into the beads 102 by immersing the beads 102 in the conductive fluid in advance.

The absorbent sheet 103 covers the container 101 together with the beads 102, and thus the beads 102 are housed in the container 101, as shown in FIG. 1. The absorbent sheet 103 may be bound to the side surface 101a with a band 103a such as rubber and thread. At this time, since the beads 102 are housed to protrude a little from the side surface 101a, as described above, the absorbent sheet 103 is fixed to the container 101 while having some tension therebetween.

The absorbent sheet 103 may be formed of a material having water-absorbing properties, e.g., a sheet-like member including a polyolefin film. As such a member, a member used as a surface member (skin contact member) of a diaper may be diverted, for example.

The biological signal detection electrode 100 is configured as described above. By configuring the biological signal detection electrode 100 as described above, the following effects can be obtained.

That is, when the biological signal detection electrode 100 comes into contact with a surface of a biological body, the conductive fluid impregnated into the beads 102 permeates through the absorbent sheet 103 and attaches to the surface of the biological body, thereby bringing the biological signal detection electrode 100 into contact with the surface of the biological body electrically. At this time, since the beads 102 individually move along a shape of the surface of the biological body in the container 101, it is possible to ensure physical contact between the absorbent sheet 103 and the surface of the biological body. In addition, the biological signal detection electrode 100 can be flexibly brought into contact with the surface of the biological body.

Furthermore, since the conductive fluid is impregnated into the beads 102 and covered with the absorbent sheet 103, the conductive fluid evaporates slowly. It is also possible to prevent the conductive fluid from drying even if the biological signal detection electrode 100 is used for a long time. Moreover, when the biological signal detection electrode 100 is removed from the surface of the biological body after a measurement is completed, it is possible to remove dirt on the surface of the biological body, which is caused due to the conductive fluid, because the absorbent sheet 103 absorbs, to some extent, the conductive fluid attached to the surface of the biological body.

### Second Embodiment

A biological signal detection electrode according to a second embodiment of the present disclosure will be described. It should be noted that, in this embodiment, a description on the same configurations as those of the biological signal detection electrode 100 according to the first embodiment will be omitted.

FIG. 4 is a perspective view showing a biological signal detection electrode 200 according to this embodiment. It should be noted that the biological signal detection electrode 200 is covered with the absorbent sheet 103 as in the biological signal detection electrode 100 according to the first embodiment, but the absorbent sheet 103 is not shown in FIG. 4.

As shown in FIG. 4, the biological signal detection electrode 200 includes a superabsorbent polymer 201 instead of the beads 102 of the biological signal detection electrode 100 according to the first embodiment.

The superabsorbent polymer 201 is processed in size and shape capable of being housed in the container 101 and is housed in the container 101 when being used. Favorably, the superabsorbent polymer 201 has a size that protrudes a little from the side surface 101a of the container 101. The superabsorbent polymer 201 may be formed of a cross-linked polyacrylic acid partial sodium salt, and a superabsorbent polymer used for a diaper may be diverted as the superabsorbent polymer 201.

The superabsorbent polymer 201 is impregnated with the conductive fluid when being used. Although the conductive fluid is not particularly limited, for example, sodium chloride (0.9%) or an electrode jelly (e.g., Signagel® electrode gel) may be used. The conductive fluid can be impregnated into the superabsorbent polymer 201 by mixing with the superabsorbent polymer 201 in advance.

The absorbent sheet 103 (not shown in FIG. 4) may be a material having water-absorbing properties, e.g., a sheet-like member including a polyolefin film, as in the first embodiment. The absorbent sheet 103 covers the container 101 together with the superabsorbent polymer 201 and thus the superabsorbent polymer 201 is housed in the container 101. At this time, since the superabsorbent polymer 201 is formed so as to have the size that protrudes a little from the side surface 101a, as described above, the absorbent sheet 103 is fixed to the container 101 while having some tension.

The biological signal detection electrode 200 is configured as described above. By configuring the biological signal detection electrode 200 as described above, the following effects can be obtained.

That is, when the biological signal detection electrode 200 comes into contact with a surface of a biological body, the conductive fluid impregnated into the superabsorbent polymer 201 permeates through the absorbent sheet 103 and attaches to the surface of the biological body, thereby bringing the biological signal detection electrode 200 into contact with the surface of the biological body electrically. At this time, since the superabsorbent polymer 201 is deformed along a shape of the surface of the biological body in the container 101, it is possible to ensure physical contact between the absorbent sheet 103 and the surface of the biological body. In addition, the biological signal detection electrode 200 can be flexibly brought into contact with the surface of the biological body.

Furthermore, since the conductive fluid is impregnated into the superabsorbent polymer 201 and covered with the absorbent sheet 103, the conductive fluid evaporates slowly. It is also possible to prevent the conductive fluid from drying even if the biological signal detection electrode 200 is used for a long time. Moreover, when the biological signal detection electrode 200 is removed from the surface of the biological body after a measurement is completed, it is possible to remove dirt on the surface of the biological body, which is caused due to the conductive fluid, because the absorbent sheet 103 absorbs, to some extent, the conductive fluid attached to the surface of the biological body.

### Third Embodiment

A biological signal detection electrode according to a third embodiment of the present disclosure will be described. It should be noted that, in this embodiment, a description on the same configurations as those of the biological signal detection electrode 100 according to the first embodiment will be omitted.

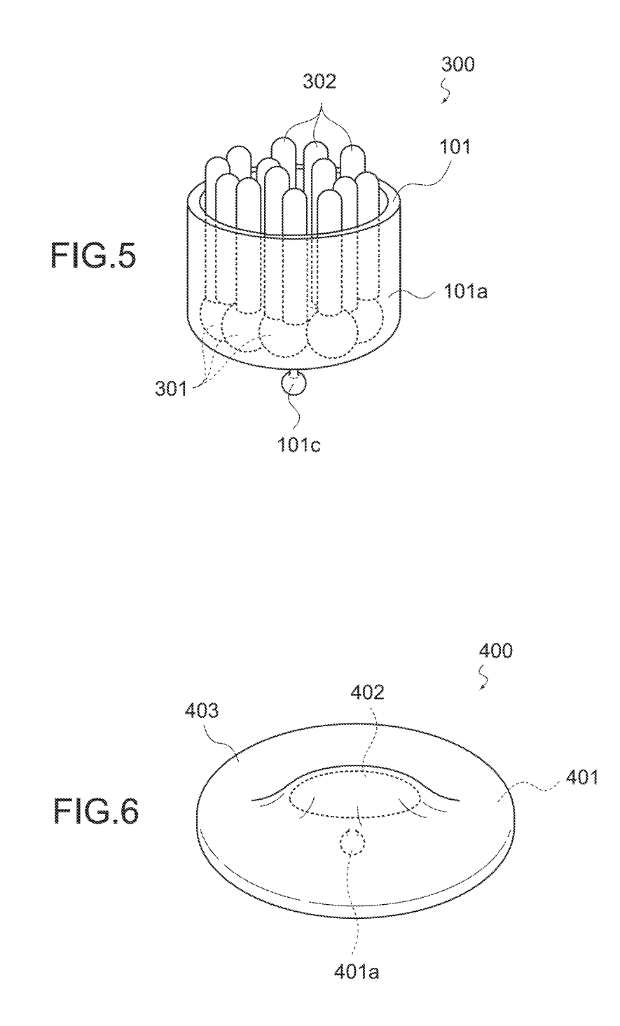

FIG. 5 is a perspective view showing a biological signal detection electrode 300 according to this embodiment. It should be noted that the biological signal detection electrode 300 is covered with the absorbent sheet 103 as in the biological signal detection electrode 100 according to the first embodiment, but the absorbent sheet 103 is not shown in FIG. 5.

As shown in FIG. 5, the biological signal detection electrode 300 includes beads 301 and cotton applicators 302 instead of the beads 102 of the biological signal detection electrode 100 according to the first embodiment.

Although the beads 301 may be formed of a water-retentive material as in the beads 102 according to the first embodiment and have the same size as the beads 102 according to the first embodiment, the beads 301 in number capable of being lined on the bottom surface 101b of the container 101 so as not to be overlaid on each other are housed, unlike the first embodiment.

The cotton applicators 302 may be general cotton applicators having cut to appropriate size. The cotton applicators 302 are placed vertically to the bottom surface 101b of the container 101 on the beads 301 arranged as described above. Specifically, the cotton applicators 302 in number that fills the volume of the container 101 are used. The cotton applicators 302 have a size that protrudes a little from the side surface 101a.

The beads 301 and the cotton applicators 302 are impregnated with the conductive fluid as in the first embodiment. Alternatively, the conductive fluid may be impregnated into only the beads 301. Also in this case, the conductive fluid is supplied from the beads 301 to the cotton applicators 302.

The absorbent sheet 103 (not shown in FIG. 5) may be a material having water-absorbing properties, e.g., a sheet-like member formed of a polyolefin film, as in the first embodiment. The absorbent sheet 103 covers the container 101 together with the cotton applicators 302 and the beads 301, and thus the beads 301 and the cotton applicators 302 are housed in the container 101. At this time, since the cotton applicators 302 have the size that protrudes a little from the side surface 101a as describe above, the absorbent sheet 103 is fixed to the container 101 while having some tension.

The biological signal detection electrode 300 is configured as described above. By configuring the biological signal detection electrode 300 as described above, the following effects can be obtained.

That is, when the biological signal detection electrode 300 comes into contact with a surface of a biological body, the conductive fluid impregnated into the beads 301 and the cotton applicators 302 permeates through the absorbent sheet 103 and attaches to the surface of the biological body, thereby bringing the biological signal detection electrode 300 into contact with the surface of the biological body electrically. At this time, since the cotton applicators 302 stretch along a shape of the surface of the biological body while rotating on the beads 301, it is possible to ensure physical contact between the absorbent sheet 103 and the surface of the biological body. In addition, the biological signal detection electrode 300 can be flexibly brought into contact with the surface of the biological body.

Furthermore, since the conductive fluid is impregnated into the beads 301 and the cotton applicators 302 and covered with the absorbent sheet 103, the conductive fluid evaporates slowly. It is also possible to prevent the conductive fluid from drying even if the biological signal detection electrode is used for a long time. Moreover, when the biological signal detection electrode 300 is removed from the surface of the biological body after a measurement is completed, it is possible to remove dirt on the surface of the biological body, which is caused due to the conductive fluid, because the absorbent sheet 103 absorbs, to some extent, the conductive fluid attached to the surface of the biological body.

### Fourth Embodiment

A biological signal detection electrode according to a fourth embodiment of the present disclosure will be described.

FIG. 6 is a perspective view showing a biological signal detection electrode 400 according to this embodiment. As shown in the figure, the biological signal detection electrode 400 includes a disposable electrode 401, a conductive gel 402, and an absorbent sheet 403. The conductive gel 402 is placed on the disposable electrode 401, and the absorbent sheet 403 covers the conductive gel 402.

The disposable electrode 401 is an electrode normally used alone by adhering to human skin or the like and includes a skin adhesive surface applied with a conductive adhesive gel. FIG. 6 shows a side of the skin adhesive surface of the disposable electrode 401. On the rear side of the skin adhesive surface, a terminal 401a for connecting the biological signal detection electrode 400 to a brace (e.g., headgear) is provided. The biological signal detection electrode 400 according to this embodiment is formed by using this disposable electrode 401.

As the conductive gel 402, for example, Signagel® electrode gel may be used, and the conductive gel 402 is placed on the center of the skin adhesive surface. Although the amount of the conductive gel 402 is arbitrary, favorably the conductive gel 402 has an amount that a convex portion is formed on the center of the skin adhesive surface.

The absorbent sheet 403 may be a material having water-absorbing properties, e.g., a sheet-like member formed of a polyolefin film, as in the first embodiment. The absorbent sheet 403 covers the skin adhesive surface together with the conductive gel 402. The absorbent sheet 403 through which the conductive gel 402 can permeate may be used. The absorbent sheet 403 may be fixed to the disposable electrode 401 with thread or the like (not shown).

The biological signal detection electrode 400 is configured as described above. By configuring the biological signal detection electrode 400 as described above, the following effects can be obtained.

That is, when the biological signal detection electrode 400 comes into contact with a surface of a biological body, the conductive gel 402 permeates through the absorbent sheet 403 and attaches to the surface of the biological body, thereby bringing the biological signal detection electrode 400 into contact with the surface of the biological body electrically. At this time, since the conductive gel 402 is deformed along a shape of the surface of the biological body, it is possible to ensure physical contact between the absorbent sheet 403 and the surface of the biological body. In addition, the biological signal detection electrode 400 can be flexibly brought into contact with the surface of the biological body.

Furthermore, since the conductive gel 402 is covered with the absorbent sheet 403, the conductive gel 402 is prevented from drying. Moreover, when the biological signal detection electrode 400 is removed from the surface of the biological body after a measurement is completed, it is possible to remove dirt on the surface of the biological body, which is caused due to the conductive gel 402 because the absorbent sheet 403 absorbs, to some extent, the conductive gel 402 attached to the surface of the biological body.

### Fifth Embodiment

A biological signal detection electrode according to a fifth embodiment of the present disclosure will be described.

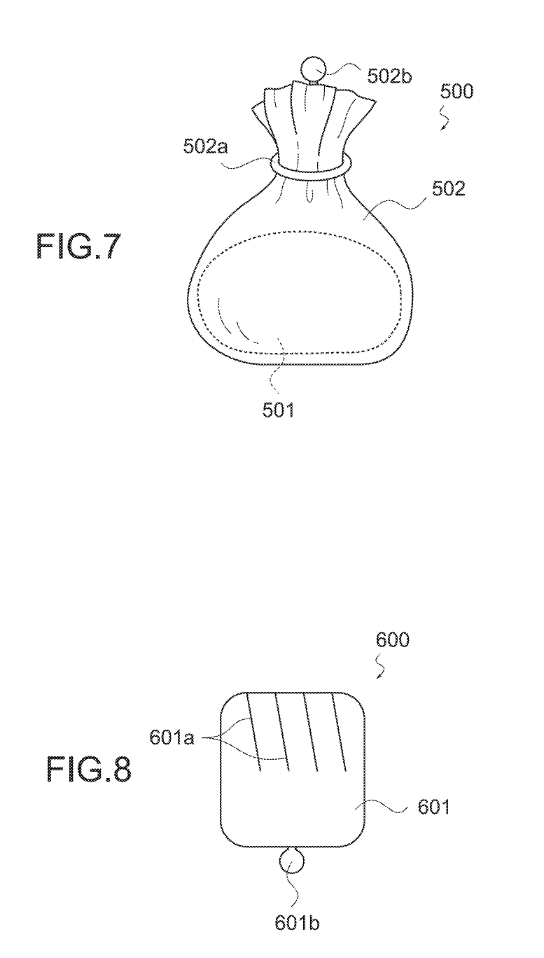

FIG. 7 is a perspective view showing a biological signal detection electrode 500 according to this embodiment. As shown in the figure, the biological signal detection electrode 500 includes a conductive gel 501 and an absorbent sheet 502 and has a configuration that the conductive gel 501 is covered with the absorbent sheet 502.

As the conductive gel 501, Signagel® electrode gel may be used, and the amount of the conductive gel 501 is arbitrary.

The absorbent sheet 502 may be a material having water-absorbing properties, e.g., a sheet-like member formed of a polyolefin film, as in the first embodiment and covers the conductive gel 501. The absorbent sheet 502 through which the conductive gel 501 can permeate may be used. The absorbent sheet 502 may be bound, with a band 502a, to be shaped liked a bag.

To the absorbent sheet 502, a terminal 502b for connecting the biological signal detection electrode 500 to a brace (e.g., headgear) is provided. The terminal 502b may be inserted into the absorbent sheet 502 and brought into contact with the conductive gel 501.

The biological signal detection electrode 500 is configured as described above. By configuring the biological signal detection electrode 500 as described above, the following effects can be obtained.

That is, when the biological signal detection electrode 500 comes into contact with a surface of a biological body, the conductive gel 501 permeates through the absorbent sheet 502 and attaches to the surface of the biological body, thereby bringing the biological signal detection electrode 500 into contact with the surface of the biological body electrically. At this time, since the conductive gel 501 is deformed along a shape of the surface of the biological body, it is possible to ensure physical contact between the absorbent sheet 502 and the surface of the biological body. In addition, the biological signal detection electrode 500 can be flexibly brought into contact with the surface of the biological body.

Furthermore, since the conductive gel 501 is covered with the absorbent sheet 502, the conductive gel 501 is prevented from drying. Moreover, when the biological signal detection electrode 500 is removed from the surface of the biological body after a measurement is completed, it is possible to remove dirt on the surface of the biological body, which is caused due to the conductive gel 501 because the absorbent sheet 502 absorbs, to some extent, the conductive gel 501 attached to the surface of the biological body.

### Sixth Embodiment

A biological signal detection electrode according to a sixth embodiment of the present disclosure will be described.

FIG. 8 is a perspective view showing a biological signal detection electrode 600 according to this embodiment. As shown in the figure, the biological signal detection electrode 600 includes a sponge 601.

As the sponge 601, a general household sponge may be used, and the size of the sponge 601 may be selected as appropriate. It should be noted that there are cuts 601a on the sponge 601. The cuts 601a may be formed in a lattice shape in the middle of the sponge 601, for example. Moreover, on the sponge 601, a terminal 601b for connecting the biological signal detection electrode 600 to a brace (e.g., headgear) is provided.

Into the sponge 601, conductive fluid such as sodium chloride (0.9%) or a conductive gel such as Signagel® electrode gel is impregnated. It should be noted that the sponge 601 is favorably a fine sponge in order to prevent the conductive fluid from drying.

The biological signal detection electrode 600 is configured as described above. By configuring the biological signal detection electrode 600 as described above, the following effects can be obtained.

That is, when the biological signal detection electrode 600 comes into contact with a surface of a biological body, the conductive fluid impregnated into the sponge 601 is attached to the surface of the biological body, thereby bringing the biological signal detection electrode 600 into contact with the surface of the biological body electrically. At this time, since the cuts 601a are formed to the sponge 601, it is possible to ensure physical contact between the sponge 601 and the surface of the biological body. In addition, the biological signal detection electrode 600 can be flexibly brought into contact with the surface of the biological body.

Each of the biological signal detection electrodes described in the first to sixth embodiments can be attached to a brace (e.g., headgear) for bringing each of the biological signal detection electrodes into contact with the surface of the biological body and used as a biological signal detection apparatus.

The present disclosure is not limited to the above-mentioned embodiments, and various modifications can be made without departing from the gist of the present technology.

It should be noted that the present disclosure may also employ the following configurations.

(1) A biological signal detection electrode, including:

a water-containing member that is impregnated with conductive fluid and has flexibility; and

an absorbent sheet that covers the water-containing member and through which the conductive fluid is capable of permeating.

(2) The biological signal detection electrode according to (1), further including

a container that is formed of a side surface and a bottom surface and includes an electrode terminal having conduction to the bottom surface, in which

the water-containing member is housed in the container, and

the absorbent sheet covers the container together with the water-containing member.

(3) The biological signal detection electrode according to (1) or (2), in which

the water-containing member is formed of a plurality of beads.

(4) The biological signal detection electrode according to any one of (1) to (3), in which

the water-containing member is formed of a superabsorbent polymer.

(5) The biological signal detection electrode according to any one of (1) to (4), in which

the water-containing member is formed of a plurality of beads arranged on the bottom surface of the container and a plurality of cotton applicators arranged on the plurality of beads.

(6) The biological signal detection electrode according to any one of (1) to (5), in which

the absorbent sheet is formed of a polyolefin film. (7) The biological signal detection electrode according to any one of (1) to (6), in which

the water-containing member is formed of a cross-linked polyacrylic acid partial sodium salt.

(8) A biological signal detection apparatus, including:

a biological signal detection electrode including a water-containing member that is impregnated with conductive fluid and has flexibility and an absorbent sheet that covers the water-containing member and through which the conductive fluid is capable of permeating; and

a brace configured to bring the biological signal detection electrode into contact with a biological body.

The present disclosure contains subject matter related to that disclosed in Japanese Priority Patent Application JP 2011-219418 filed in the Japan Patent Office on Oct. 3, 2011, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A biological signal detection electrode, comprising:
a water-containing member that is impregnated with conductive fluid and has flexibility; and
an absorbent sheet that covers the water-containing member and through which the conductive fluid is capable of permeating.

2. The biological signal detection electrode according to claim 1, further comprising
a container that is formed of a side surface and a bottom surface and includes an electrode terminal having conduction to the bottom surface, wherein
the water-containing member is housed in the container, and
the absorbent sheet covers the container together with the water-containing member.

3. The biological signal detection electrode according to claim 2, wherein
the water-containing member is formed of a plurality of beads.

4. The biological signal detection electrode according to claim 2, wherein
the water-containing member is formed of a superabsorbent polymer.

5. The biological signal detection electrode according to claim 2, wherein
the water-containing member is formed of a plurality of beads arranged on the bottom surface of the container and a plurality of cotton applicators arranged on the plurality of beads.

6. The biological signal detection electrode according to claim 1, wherein
the absorbent sheet is formed of a polyolefin film.

7. The biological signal detection electrode according to claim 1, wherein
the water-containing member is formed of a cross-linked polyacrylic acid partial sodium salt.

8. A biological signal detection apparatus, comprising:
a biological signal detection electrode including a water-containing member that is impregnated with conductive fluid and has flexibility and an absorbent sheet that covers the water-containing member and through which the conductive fluid is capable of permeating; and
a brace configured to bring the biological signal detection electrode into contact with a biological body.
